(19) United States
(12) Patent Application Publication
(10) Pub. No.: US 2015/0051558 A1
(43) Pub. Date: Feb. 19, 2015

(54) COMPOSITION AND METHOD FOR CORNEAL PROLIFERATION

(71) Applicant: UNIVERSITY OF CENTRAL FLORIDA RESEARCH FOUNDATION, INC., Orlando, FL (US)

(72) Inventor: Kiminobu Sugaya, Winter Park, FL (US)

(21) Appl. No.: 14/389,398

(22) PCT Filed: Mar. 13, 2013
(86) PCT No.: PCT/US13/30826

§ 371 Date: Sep. 30, 2014

Related U.S. Application Data
(60) Provisional application No. 61/620,915, filed on Apr. 5, 2012.

(51) Int. Cl. A61K 31/519; A61K 9/00; A61F 9/00

(52) U.S. Cl. CPC A61K 31/519; A61F 9/0026; A61K 9/0048; A61K 9/0051
USPC 604/290; 604/295; 604/296; 514/265.1

(57) ABSTRACT

Disclosed herein in exemplary embodiments are methods and compositions for repairing or treating a defected cornea in a patient in need. The methods and compositions including, for example, administering a dose of a composition to the patient, wherein the composition comprises may be administered to an intraocular area of the patient via a container having a spout for ophthalmic delivery. The methods and compositions promote progenitor cell migration and increase cellular proliferation in the cornea of the patient.

### BACKGROUND

The cornea is an organ, a transparent layer of tissue at the front of the eye which protects the intraocular contents and serves as a major optical element of the eye. The cornea is composed almost entirely of a special type of collagen. Normally, no blood vessels are found in the cornea, but because it contains nerve endings, cornea damage can be very painful. Seventy-five percent of the diopteric power of the eye depends on the interface of the cornea and the air. Injury, disease, or cellular failure can cause opacification of the cornea with subsequent impairment and corneal blindness. Corneal opacification affects more than 10 million patients worldwide, and is often treated by transplantation of deceased donor tissues, or the more recently developed stem cell biopsy and transplant methods. These transplantation procedures often have a poor success rate due in part to issues such as rejection of donor tissues, complexity of the procedures, increased demand for corneal donors coupled with decreased shelf life of donated eyes lasting only a few days. The known methods of corneal treatment and repair, including transplantation, also involve a great cost as they must be performed in a hospital or physician's office setting as they include invasive procedures, and the donated tissues are not always readily available. According to the Eye Bank Association of America, more than 40,000 corneal transplants are performed in the United States each year, and corneal transplant recipients range in age from 9 days to 103 years. In a typical corneal transplant, a disc of tissue is removed from the center of the eye and replaced by a corresponding disc from a donor eye. The circular incision is made using an instrument called a trephine, which resembles a cookie cutter. In one form of corneal transplant, penetrating keratoplasty (PK), the disc removed is the entire thickness of the cornea and so is the replacement disc. (See at: http://www.surgeryencyclopedia.com/Ce-Fi/Corneal-Transplantation.html#ixzz1r5IxCtTb). The donor cornea is attached with extremely fine sutures in the transplant. Surgery can be performed under anesthesia that is confined to one area of the body while the patient is awake (local anesthesia) or under anesthesia that places the entire body of the patient in a state of unconsciousness (general anesthesia). Corneal transplant surgery typically requires 30-90 minutes. Over 90% of all corneal transplants in the United States are PK. In lamellar keratoplasty (LK), only the outer layer of the cornea is removed and replaced. LK has many advantages, including early suture removal and decreased infection risk. It is not as widely used as PK, however, because it is more time consuming and requires much greater technical ability by the surgeon.

Keratoplasty is the most common type of human transplant surgery and boasts the highest success rate. Corneal transplants are often required when a patient has lost their vision due to cornea damage as a result of disease or injury, and no other viable options exist. Corneal blindness is a cause of 8-25% of blindnes sin developing countries (Garg et al., Cambridge Ophthalmological Symposium, Eye (2005) 19, 1106-1114. doi:10.1038/sj.eye.6701968 “The value of corneal transplantation in reducing blindness”). Various corneal conditions which cause cloudiness of the cornea or alter the natural curvature of the organ and which can reduce vision quality include keratoconus (outward bulging of the cornea), Fuchs' dystrophy (malfunction of the cornea's inner layer), psudophakic bullous keratopathy (painful corneal swelling), pterygium (tissue growth on the cornea), and Stevens-Johnson syndrome (skin disorder affecting the eyes) among others. These various diseases may require a corneal transplant. A corneal transplant may also be required where injury to the cornea occurs due to chemical burns, mechanical trauma, or infection by viruses, bacteria, fungi, or protozoa.

Ultimately, the risks and costs associated with corneal transplantation procedures including issues such as rejection of donor tissues, complexity of procedures, increased demand for corneal donors coupled with decreased shelf life of donated eyes lasting only a few days, increased costs involved wherein specialized surgeons or the use of medical facilities are required, as well as reliability on donated tissues demonstrates a need for a less invasive, more cost-effective method of treatment.

### SUMMARY

Discovered herein are methods and compositions which provide an increase in cellular proliferation of the cornea. In contrast to the pricey and complex current treatment methods for corneal repair or replacement including transplants from deceased donors and stem cell biopsy and transplant methods, the subject methods and compositions are not prohibitive. Further, the level of invasiveness of the subject embodiments is far less than that of the current standard of practice and treatment. Embodiments disclosed herein provide rapid, substantial, cornea re-growth in patients treated by the methods and compositions discovered herein. Exponential corneal cell proliferation has been achieved by the methods and compositions described herein.

### BRIEF DESCRIPTION

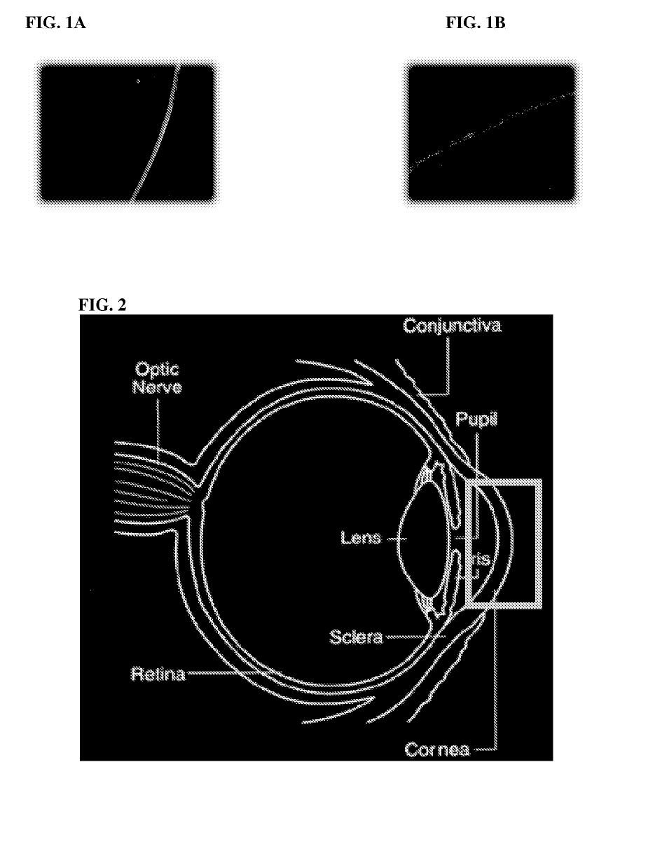

FIG. 1A provides a view of a 100× magnification of a 20 micron thick saggital slice of the cornea of a control animal, which received eye drop containing 0 mg/ml MS-818. Red dots represent BrdU immunoreactivity indicating proliferating cells. Blue is counter staining of the cell nuclei.

FIG. 1B provides a view of a 100× maginification of a 20 micron thick saggital slice of the cornea of a high dosage animal, which received eye drop containing 1 mg/ml MS-818. Red dots represent BrdU immunoreactivity indicating proliferating cells. Blue is counter staining of the cell nuclei.

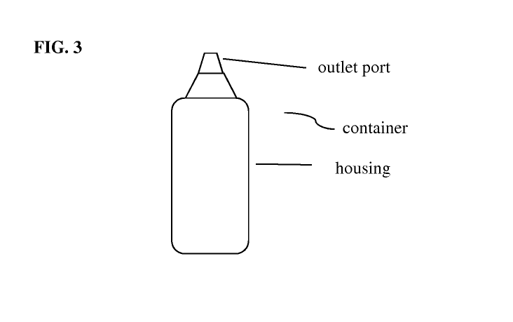

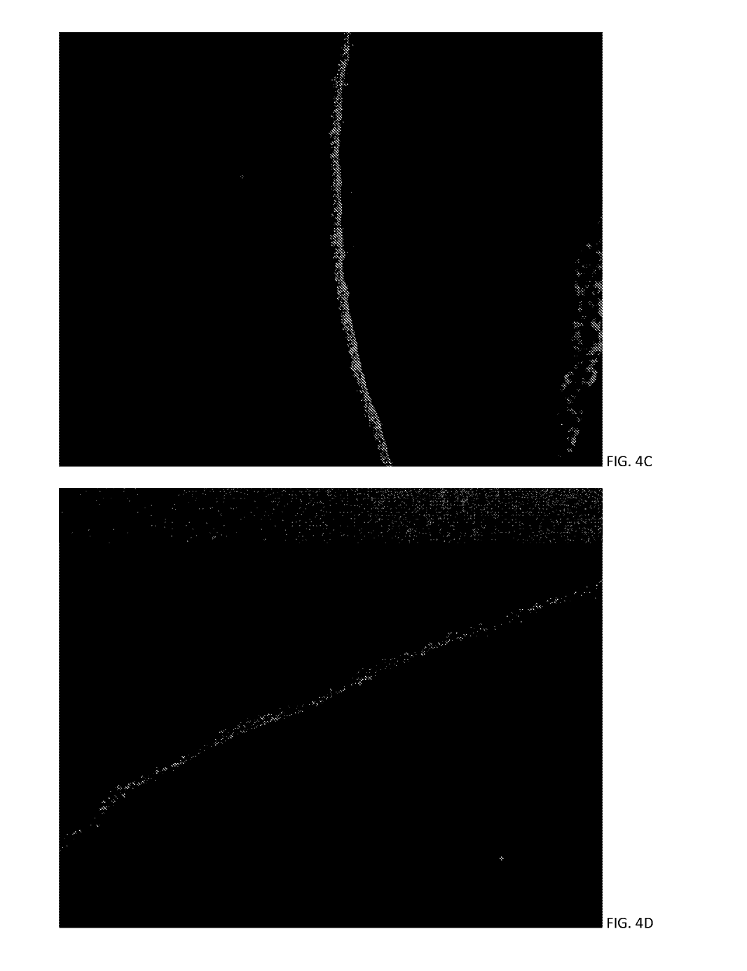

FIG. 2 provides a schematic of a saggital section of an eye, wherein the field of view of FIGS. 1 and 4 images are highlighted in the blue box. The image was obtained from the National Eye Institute website, on the Facts About the Cornea and Corneal Disease page at http://www.nei.nih.gov/health/cornealdisease/#6.

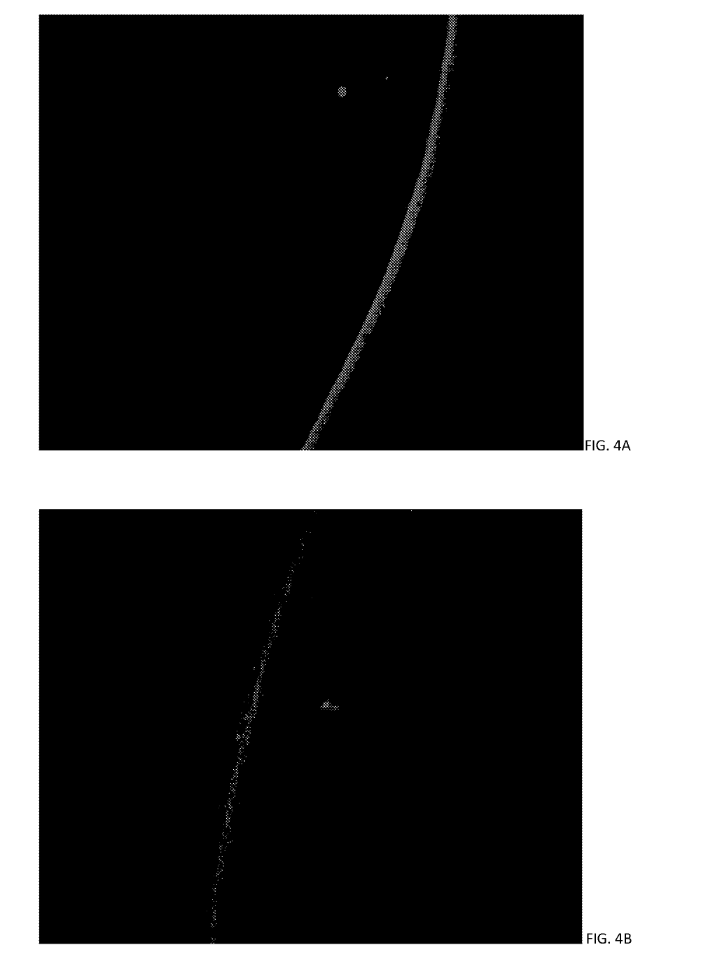

FIG. 3 is a diagram of one example of a container configured for dispensing eye drops. The container includes a housing for holding a volume of composition for delivery to the eye, and an outlet port.

FIG. 4A-D show a series of photographs of sagittal slices of rat eyes showing the presence of BrdU (red) which correlates to increased cell proliferation. FIG. 4A relates to the control animal, FIG. 4B relates to an animal treated with 100 micrograms/ml, FIG. 4C relates to an animal treated with 300 micrograms/ml, and FIG. 4D relates to animals treated with 1000 micrograms/ml.

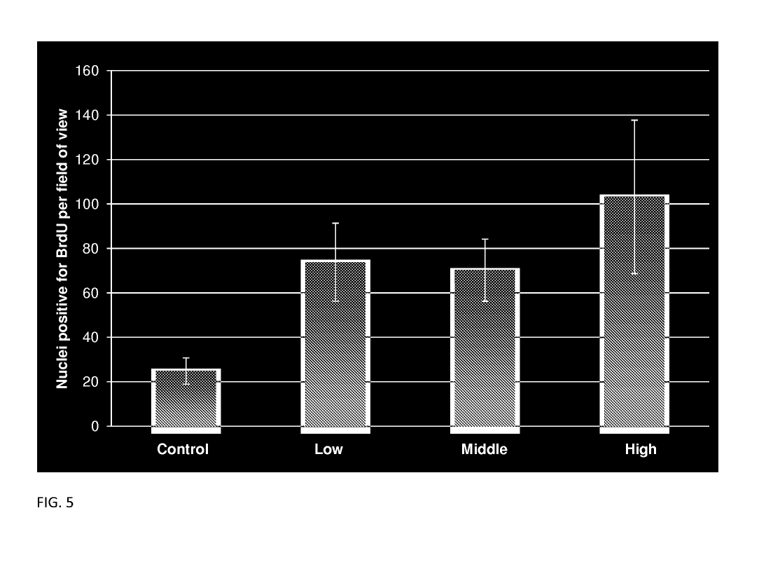

FIG. 5 represents number of nuclei positive for BrdU counted in the cornea of several different animals (n=6) similar to the field of view shown in FIG. 4. Vertical columns represent mean of control low dose, medium does and high dose of MS-818 and vertical lines on the each column represent standard deviations of each group. Numbers of BrDU positive nuclei in the treated group are significantly (p<0.05) higher than the control.

### DETAILED DESCRIPTION

For the purposes of promoting an understanding of the principles and operation of the invention, reference will now be made to the embodiments illustrated in the drawings and specific language will be used to describe the same. It will nevertheless be understood that no limitation of the scope of the invention is thereby intended, such alterations and further modifications in the illustrated methods and devices, and such further applications of the principles of the invention as illustrated therein being contemplated as would normally occur to those skilled in the art to which the invention pertains.

It is demonstrated herein for the first time, the ability of a proprietary ophthalmic solution to increase cellular proliferation in a cornea. Clinical indications for the re-growth of healthy cornea tissue are numerous, including conditions implicated in blindness. Current known methods of treatment for these conditions include cornea transplants from deceased donors as well as the more recently developed stem cell biopsy and transplant method. Both price and complexity are prohibitive for the use of these treatments in many populations. The technology disclosed herein provides a far less invasive, far easier to administer, and more cost effective alternative to these treatments. As discussed in greater detail below, animals receiving the solution demonstrated exponential corneal cell proliferation over control animals, representing clinical efficacy in regard to achieving rapid and substantial cornea re-growth.

The embodiments herein provide a novel invention which supplants the more invasive and costly treatments available for corneal injury or disease. Currently, a diverse group of corneal conditions are indications for corneal transplants or limbal transplants (transplantation of stem cells with tissue from the corneoscleral limbus). According to the National Eye Institute, these conditions include Fuch's dystrophy, iridocorneal endothelial syndrome, keratoconus, and corneal scarring, among others (see at www.nei.nih.gov). Corneal transplants and allo-limbal transplants both require the use of donor tissue. These treatments are followed by a life time of immunosuppressive therapy to avoid graft rejection. The administration of MS-818 via ophthalmic drops, in one example, provides a simple and cost effective alternative to these treatments which increase treatment availability and decrease side effects of the alternative treatments including possible graft rejection.

In an embodiment, a method of treating a condition characterized by a corneal defect is provided. The method includes applying a composition comprising MS-818 (2-piperadino-6-methyl-5-oxo-5,6-dihydro-(7H) pyrrole-[3,4-d]pyrimidine maleate) or a pharmaceutically acceptable salt of the pyrimidine compound other than a maleate salt. The condition being treated may include Fuch's dystrophy, iridocorneal endothelial syndrome, keratoconus, corneal scarring, Stephen Johnson's syndrome, pterygium, and/or keratitis. In one embodiment, the method is provided wherein the composition is applied to an intraocular area of the subject in need.

Examples of other pharmaceutically-acceptable salts of the compound include salts formed from acids capable of forming pharmaceutically-acceptable non-toxic acid-addition salts containing anions, such as the hydrochloride, hydrobromide, sulfate, bisulfite, phosphate, acid phosphate, acetate, maleate, fumarate, succinate, lactate, tartrate, benzoate, citrate, gluconate, glucanate, methanesulfonate, p-toluenesulfonate and naphthalenesulfonate, and their hydrates, as well as the quaternary ammonium (or amine) salt and its hydrate.

In another embodiment, the method is provided wherein applying the composition to the subject includes providing a container including a composition including MS-818; aligning an outlet port of the container with an eye of the subject in need; and ejecting the composition via the outlet port of the container to an intraocular area of the subject in need.

In another embodiment, a method of repairing a cornea or a portion thereof in a patient in need is provided. The method includes administering a composition to the patient, wherein the composition comprises MS-818 (2-piperadino-6-methyl-5-oxo-5,6-dihydro-(7H) pyrrole-[3,4-d]pyrimidine maleate). The method further includes wherein the composition is administered to an intraocular area of the patient. In a further embodiment, the patient in need exhibits a corneal injury. In still a further embodiment, the composition is a liquid or a semi-solid.

In yet another embodiment, the method of repairing a cornea or a portion thereof in a patient in need is provided, including providing a container comprising a composition comprising MS-818; aligning an outlet port of the container with an eye of the patient in need; and administering the composition via the outlet port of the container to an intraocular area of the patient in need.

In still another embodiment, there is provided a container including a composition that includes MS-818, wherein the container includes an outlet port for ophthalmic delivery. The container is further provided wherein the composition is a liquid or a semi-solid. In a further embodiment, the container is provided wherein the outlet port includes a spout. In still a further embodiment, the container includes a non-aerosol, non electric delivery mechanism for ophthalmic delivery in the form of a spray or a mist. Examples of ophthalmic delivery devices include, but are not limited to, those taught in U.S. Patent Pub. Nos. 2004/0052877; 2005/0165368; 2008/0233052; U.S. Pat. No. 4,484,922 (membrane that could be infused with MS818 or related salt and placed over the cornea where the compound is contacted with cells of the cornea), U.S. Pat. Nos. 4,733,802; 6,736,802; 6,740,065; 6,506,183; 5,059,188, 4,834,728; 4,960,407; and 3,756,478.

In a further embodiment, a pharmaceutical composition for the treatment of a corneal defect in a subject in need is provided. The pharmaceutical composition includes a therapeutic agent, wherein the therapeutic agent includes MS-818. The MS-818 promotes progenitor cell migration and increases cellular proliferation in the cornea of the subject when the pharmaceutical composition is administered to an eye area of the subject. The pharmaceutical composition is provided as a liquid or a semi-solid in one embodiment. In a further embodiment, the composition is administered to an intraocular area of an eye of the subject. In still a further embodiment, the composition comprises a solution for ophthalmic delivery.

### EXAMPLES

### Effect of MS-818 in Cornea Regeneration

MS-818 (2-piperadino-6-methyl-5-oxo-5,6-dihydro-(7H) pyrrole-[3,4-d]pyrimidine maleate) was discovered herein for its capacity to promote proliferation of endogenous stem cells in host organisms. Broad proliferative effects have been identified herein in multiple tissue types. Further, MS-818's effect on tissue regeneration in the cornea has never heretofore been discovered. In a series of controlled experiments, MS-818 was administered via ophthalmic drops to animals divided into three dosage groups and a control group. MS-818 was tested for its effect on cornea regeneration both in the presence and absence of injury. Preliminary results show a marked increase in proliferation in the non-injured eyes for those animals exposed to the drug over those in the non-drug receiving control group. Therefore, it has been identified herein that subjects with a broad range of corneal diseases or injuries benefit from the use of this non-invasive corneal treatment.

### Experiment 1-Materials and Methods

Surgeries were performed to remove the left lachrymal gland from sixteen rats. MS-818 was then administered via ophthalmic drop to both eyes of twelve of the rats divided into three dosage groups, 1 mg/ml, 3 mg/ml, and 10 mg/ml. The remaining four rats served as a control group, receiving drops of phosphate buffered saline. The compound was administered three times over a period of three days. BrdU was administered during this time via intra-peritoneal injection. Rats were euthanized seven days after the first compound administration and underwent perfusion. Eyes were sliced in twenty micron slices using a cryostat. The slices were mounted on glass slides. The slides were then stained with monoclonal mouse anti BrdU primary antibody to test for the incorporation of BrdU into nuclei. TRITC conjugated donkey anti mouse secondary antibody was used to detect the primary antibody. Slides were stained with DAPI to visualize the nuclei. Images were captured of the slices in 100× magnification under TRITC, DAPI and FITC fluorescence.

### Results

Antibody staining for BrdU revealed significant differences between the non surgical (left) eye of the control and drug receiving animals. Turning to the Figures, FIGS. 1A and 1B are 100× magnifications of 20 micron thick sagittal slices of the cornea of a control animal (FIG. 1A) and a high dosage (1 mg/ml) receiving animal (FIG. 1B). The structures in each Figure are the mid-portion of the cornea. Blue signal is a counter staining of nuclei. Red fluorescence is indicative of positive BrdU staining, and consequentially a positive result for cell proliferation. FIG. 1A shows little to no BrdU positive signaling (red) within the cornea, while FIG. 1B shows significantly increased BrdU positive signaling. These findings demonstrate that MS-818 induces cellular proliferation in the rat cornea.

FIG. 2 provides a schematic of a sagittal section of the eye, field of view of FIGS. 1 and 4 images are highlighted in the blue box. Image obtained from National Eye Institute website, on the Facts About the Cornea and Corneal Disease (see at: http://www.nei.nih.gov/health/cornealdisease/#6).

### Experiment 2-Materials and Methods

According to another example similar to experiment 1, surgeries were performed to remove the left lachrymal gland from sixteen rats. MS-818 was then administered via ophthalmic drop to both eyes of twelve of the rats divided into three dosage groups, 100 μg/ml, 300 μg/ml, and 1 mg/ml. The remaining four rats served as a control group, receiving drops of phosphate buffered saline. The compound was administered three times over a period of three days. BrdU was administered during this time via intra-peritoneal injection. Rats were euthanized seven days after the first compound administration and underwent perfusion. Eyes were sliced in twenty micron slices using a cryostat. The slices were mounted on glass slides. The slides were then stained with monoclonal mouse anti BrdU primary antibody to test for the incorporation of BrdU into nuclei. TRITC conjugated donkey anti mouse secondary antibody was used to detect the primary antibody. Slides were stained with DAPI to visualize the nuclei. Images were captured of the slices in 100× magnification under TRITC, DAPI and FITC fluorescence.

### Results

Antibody staining for BrdU revealed significant differences between the non surgical (left) eye of the control and drug receiving animals. Turning to the Figures, FIG. 4A is a sagittal eye slice of a control animal, FIG. 4B is a sagittal slice of an animal given a dosage of 100 μg/ml, FIG. 4C is a sagittal slice of an animal given a dosage of 300 μg/ml, and FIG. 4D is a sagittal slice of an animal given a 1 mg/ml dosage of MS-818 eye drop. The structures in each Figure are the mid-portion of the cornea. Red fluorescence is indicative of positive BrdU staining, and consequentially a positive result for cell proliferation. Blue is a counter staining of nulei. As can be seen, the treated animals have a significant increase in cell proliferation over the control animal. These findings further demonstrate that MS-818 induces cellular proliferation in the rat cornea.

FIG. 5 shows statistical analysis of the results of nuclei positive for BrdU per field of view. As can be seen, a significant and dose dependent increase is obtained in the MS-818 eye drop treated animals, with the high dosage animals having the highest number of BrdU positive nuclei.

### Dosage

The dose administered to a subject, particularly a human, in accordance with the present invention should be sufficient to effect the desired response in the subject over a reasonable time frame. One skilled in the art will recognize that dosage will depend upon a variety of factors, including the strength of the particular compositions employed, the age, species, condition, and body weight of the subject. The size of the dose also will be determined by the route, timing and frequency of administration as well as the existence, nature, and extent of any adverse side effects that might accompany the administration of a particular composition and the desired physiological effect. It will be appreciated by one of ordinary skill in the art that various conditions or desired results, may require prolonged treatment involving multiple administrations.

Suitable doses and dosage regimens can be determined by conventional range-finding techniques known to those of ordinary skill in the art. Generally, but not necessarily, treatment is initiated with smaller dosages, which are less than the optimum dose of the compound. Thereafter, the dosage is increased by small increments until the optimum effect under the circumstances is reached.

The amount of the compound or composition of the invention administered per dose or the total amount administered per day may be predetermined or it may be determined on an individual patient basis by taking into consideration numerous factors, including the nature and severity of the patient's condition, the condition being treated, the age, weight, and general health of the patient, the tolerance of the patient to the compound, the route of administration, pharmacological considerations such as the activity, efficacy, pharmacokinetics and toxicology profiles of the compound and any secondary agents being administered, and the like. Patients undergoing such treatment will typically be monitored on a routine basis to determine the effectiveness of therapy. Continuous monitoring by the physician will insure that the optimal amount of the compound of the invention will be administered at any given time, as well as facilitating the determination of the duration of treatment. This is of particular value when secondary agents are also being administered, as their selection, dosage, and duration of therapy may also require adjustment. In this way, the treatment regimen and dosing schedule can be adjusted over the course of therapy so that the lowest amount of compound or composition that exhibits the desired effectiveness is administered and, further, that administration is continued only so long as is necessary to successfully achieve the optimum effect.

### Pharmaceutical Compositions

Various embodiments of the invention are foreseen to have valuable application as constituents of pharmaceutical preparations to treat various conditions generally defined as pathologies. Accordingly, embodiments of the invention also comprise pharmaceutical compositions comprising one or more compounds of this invention in association with a pharmaceutically acceptable carrier. Preferably these compositions are in unit dosage forms such as ophthalmic solutions, but they may also include tablets, pills, capsules, powders, granules, sterile parenteral solutions or suspensions, metered aerosol or liquid sprays, drops, ampoules, or auto-injector devices; for ophthalmic administration, and most preferably the compositions are in unit dosage forms for ophthalmic solutions. For preparing liquid or semi-solid solutions and compositions such as the compositions identified herein, the principal active ingredient is mixed with a pharmaceutical carrier, and/or pharmaceutical diluents, e.g. water, to form a preformulation composition containing a homogeneous mixture of a compound/composition of the present invention, or a pharmaceutically acceptable equivalent thereof. When referring to these preformulation compositions as homogeneous, it is meant that the active ingredient is dispersed evenly throughout the composition so that the composition may be readily subdivided into equally effective unit dosage forms. This liquid or semi-solid preformulation composition is then subdivided into unit dosage forms of the type described above. The compositions may be contained in a vial, sponge, syringe, tube, or other suitable container.

Examples of compositions specifically adapted for ophthalmic delivery, and which could be adapted to include MS818 or related salt as the therapeutic compound, include, but are not limited to, those taught in U.S. Pat. Nos. 5,141,928; 5,776,445; 5,200,180; 5,422,116; 5,888,492; 4474751; 4,003,991, 3,450,814; and/or 3,415,929 The compositions may take the form of suspensions, solutions or emulsions in oily or aqueous vehicles and may contain formulatory agents such as suspending, stabilizing and/or dispersing agents. Alternatively the compositions may be in powder form (e.g., lyophilized) for constitution with a suitable vehicle, for example sterile pyrogen-free water, before use. Still other routes of administration may be used.

The term “co-administration” or “co-administering” as used herein refer to the administration of a substance before, concurrently, or after the administration of another substance such that the biological effects of either substance synergistically overlap.

As used herein, the term “area” or “region” includes but is not limited to the portion directly in contact with the solution, but also includes the surrounding area, including but not limited to the entire eye orbit encompassed anteriorly and posteriorly by the frontal bone and the maxilla and the zygomatic bones.

“Container” as used herein refers to a housing for a compound or composition, and includes but is not limited to: ampoules, aerosol cans, sponges, syringes, vials, tubes, bottles, pouches, eye contacts infused with agent, and strips.

As used herein, the terms “subject” and “patient” are used interchangeably. As used herein, the term “subject” refers to an animal, preferably a mammal such as a non-primate (e.g., cows, pigs, horses, cats, dogs, rats etc.) and a primate (e.g., monkey and human), and most preferably a human.

Although more than one route can be used to administer a particular compound, a particular route can provide a more immediate and more effective reaction than another route. Accordingly, the described routes of administration are merely exemplary and are in no way limiting.

It should be borne in mind that all patents, patent applications, patent publications, technical publications, scientific publications, and other references referenced herein are hereby incorporated by reference in this application in order to more fully describe the state of the art to which the present invention pertains.

Reference to particular buffers, media, reagents, cells, culture conditions and the like, or to some subclass of same, is not intended to be limiting, but should be read to include all such related materials that one of ordinary skill in the art would recognize as being of interest or value in the particular context in which that discussion is presented. For example, it is often possible to substitute one buffer system or culture medium for another, such that a different but known way is used to achieve the same goals as those to which the use of a suggested method, material or composition is directed.

It is important to an understanding of the present invention to note that all technical and scientific terms used herein, unless defined herein, are intended to have the same meaning as commonly understood by one of ordinary skill in the art. The techniques employed herein are also those that are known to one of ordinary skill in the art, unless stated otherwise. For purposes of more clearly facilitating an understanding the invention as disclosed and claimed herein, the following definitions are provided.

While a number of embodiments of the present invention have been shown and described herein in the present context, such embodiments are provided by way of example only, and not of limitation. Numerous variations, changes and substitutions will occur to those of skill in the art without materially departing from the invention herein. For example, the present invention need not be limited to best mode disclosed herein, since other applications can equally benefit from the teachings of the present invention. Also, in the claims, means-plus-function and step-plus-function clauses are intended to cover the structures and acts, respectively, described herein as performing the recited function and not only structural equivalents or act equivalents, but also equivalent structures or equivalent acts, respectively. Accordingly, all such modifications are intended to be included within the scope of this invention as defined in the following claims, in accordance with relevant law as to their interpretation.

While one or more embodiments of the present invention have been shown and described herein, such embodiments are provided by way of example only. Variations, changes and substitutions may be made without departing from the invention herein. Accordingly, it is intended that the invention be limited only by the spirit and scope of the appended claims. The teachings of all references cited herein are incorporated in their entirety to the extent not inconsistent with the teachings herein.

What is claimed is:

1. A method of treating a condition characterized by a corneal defect, the method comprising:
delivering a composition comprising MS-818 (2-piperadino-6-methyl-5-oxo-5,6-dihydro-(7H) pyrrole-[3,4-d]pyrimidine maleate) or other pharmaceutically acceptable salt of the pyrimidine compound not involving maleate, to a cornea of a subject in need.

2. The method of claim 1, wherein the defect comprises: a corneal scratch or other injury, Fuch's dystrophy, iridocorneal endothelial syndrome, keratoconus, corneal scarring, Stephen Johnson's syndrome, pterygium, or keratitis, and delivering comprises intentional contact of the composition with corneal cells of the corneal defect.

3. The method of claim 1, wherein the composition is applied to an intraocular area of the subject in need.

4. The method of claim 1, wherein applying the composition to the subject comprises
obtaining a container comprising a composition, said composition comprising MS-818,
and said container comprising a housing with an outlet port;
aligning said outlet port of the container with an eye of the subject in need; and
ejecting the composition via the outlet port of the container to an intraocular area of the subject in need.

5. A method of repairing a cornea or a portion thereof in a patient in need, comprising:
administering a dose of a composition to the patient, wherein the composition comprises MS-818 (2-piperadino-6-methyl-5-oxo-5,6-dihydro-(7H) pyrrole-[3,4-d]pyrimidine maleate) or other pharmaceutically acceptable salt of the underlying pyrimidine compound not including maleate.

6. The method of claim 5, wherein the composition is administered to an intraocular area of the patient.

7. The method of claim 5, wherein the patient in need exhibits a corneal injury.

8. The method of claim 5, wherein the composition is a liquid or a semi solid.

9. The method of claim 5, wherein the method comprises:
obtaining a container comprising a composition comprising MS-818;
aligning an outlet port of the container with an eye of the patient in need; and
ejecting the composition via the outlet port of the container to an intraocular area of the patient in need.

10. A container comprising a composition, said composition comprising MS-818, wherein the container comprises an outlet port for ophthalmic delivery.

11. The container of claim 10, wherein the composition is a liquid or a semi-solid.

12. The container of claim 10, wherein the outlet port comprises a spout.

13. The container of claim 10, wherein the container further comprises a non-aerosol, non electric delivery mechanism for ophthalmic delivery in the form of a spray or a mist.

14. An article of manufacture comprising a container comprising a mechanism configured for ophthalmic delivery and a pharmaceutical composition disposed within said container, the pharmaceutical composition useful for the treatment of a corneal defect, wherein said composition comprises MS-818, or other salt formed with the underlying pyrimidine compound other than maleate.

15. The article of manufacture of claim 14, wherein the composition is a liquid or a semi-solid.

16. The article of manufacture of claim 14, wherein the composition is administered to an intraocular area of an eye of the subject.

17. The article of manufacture of claim 14, wherein the composition comprises a solution for ophthalmic delivery.

18. An article of manufacture comprising a contact for placement on the eye, wherein said contact is loaded with MS-818, or other pharmaceutically acceptable salt formed with the underlying pyrimidine compound other than maleate.
